(19) United States
(12) Patent Application Publication
(10) Pub. No.: US 2024/0423798 A1
(43) Pub. Date: Dec. 26, 2024

(54) FLEXIBLE VALVE ANCHORS

(71) Applicant: EDWARDS LIFESCIENCES CORPORATION, Irvine, CA (US)

(72) Inventor: John Richard Carpenter, Irvine, CA (US)

(21) Appl. No.: 18/827,056

(22) Filed: Sep. 6, 2024

Related U.S. Application Data
(63) Continuation of application No. PCT/US2023/013147, filed on Feb. 15, 2023.
(60) Provisional application No. 63/269,038, filed on Mar. 8, 2022.

(51) Int. Cl. A61F 2/24; A61B 17/34

(52) U.S. Cl. CPC A61F 2/2457; A61B 17/3468; A61F 2/2466; A61F 2220/0016; A61F 2220/0075; A61F 2230/0004; A61F 2230/0008; A61F 2230/0017; A61F 2230/0063

(57) ABSTRACT

A tissue anchor comprises a frame comprising at least a first pointed end and a membrane extending across an opening of the frame. The frame is configured to bend such that the first pointed end extends at least partially over the membrane.

### RELATED APPLICATION

This application is a continuation of International Patent Application No. PCT/US2023/013147, filed Feb. 15, 2023 and entitled FLEXIBLE VALVE ANCHORS, which claims priority to U.S. Application No. 63/269,038, filed Mar. 8, 2022, the disclosures of which are hereby incorporated by reference in their entirety.

### BACKGROUND

### Field

The present disclosure generally relates to the field of medical procedures and devices.

### Description of Related Art

Various medical procedures involve accessing internal anatomy of a patient through biological tissue. Some procedures can involve delivery of deployment devices into a beating heart.

### SUMMARY

Described herein are one or more methods and/or devices to facilitate puncture site and/or orientation location and execution.

Some implementations of the present disclosure relate to a tissue anchor comprising a frame comprising at least a first pointed end and a membrane extending across an opening of the frame. The frame is configured to bend such that the first pointed end extends at least partially over the membrane.

In some instances, the frame and membrane are diamond shaped. The frame and membrane may be oval-shaped. In some instances, the frame and membrane are droplet shaped.

The first pointed end may be configured to puncture a valve leaflet of a heart. In some instances, the first pointed end comprises an eyelet configured to receive a tethering suture.

In some instances, the membrane is configured to attach to a tethering suture. The frame may be configured to bend such that the frame assumes a heart-shaped form.

Some implementations of the present disclosure relate to a method comprising delivering a needle carrying two or more tissue anchors situated longitudinally within a lumen of the needle. Each of the two or more tissue anchors comprises a frame having a pointed end and a membrane extending across an opening of the frame. Each of the two or more tissue anchors is tethered to a different suture of two or more sutures. The method further comprises deploying a first tissue anchor of the two or more tissue anchors through a puncture opening in a valve leaflet of a heart and beyond a distal end of the needle, positioning the first tissue anchor such that a first membrane of the first tissue anchor covers the puncture opening, and redirecting a first pointed end of the frame of the first tissue anchor such that the pointed end extends at least partially over the first membrane.

In some instances, the method further comprises puncturing the valve leaflet using a pointed tip of the needle. The pointed tip may extend at least partially over the lumen of the needle to cause the first tissue anchor to exit the lumen at an angle with respect to the needle.

The pointed tip may have rounded edges to cause dilation of the puncture opening. In some instances, the method further comprises deploying a second tissue anchor of the two or more tissue anchors through a second puncture opening in the valve leaflet of the heart and beyond the distal end of the needle.

In some instances, redirecting the first pointed end of the frame causes the first pointed end to extend at least partially over the puncture opening.

Some implementations of the present disclosure relate to a tissue anchoring system comprising a first tissue anchor configured for delivery via a lumen of a delivery shaft to a valve leaflet of a heart. The first tissue anchor comprises a frame comprising at least a first pointed end and a membrane extending across an opening of the frame. The frame is configured to bend such that the first pointed end extends at least partially over the membrane. The anchoring system further comprises a first suture tethered to the first tissue anchor and configured to anchor to a ventricle wall.

In some instances, the frame comprises an eyelet. The first suture may be configured to form a knot through the eyelet.

The first suture may be configured to attach to the membrane. In some instances, the tissue anchoring system further comprises a second tissue anchor configured for delivery via the lumen of the delivery shaft to the valve leaflet of the heart and a second suture tethered to the second tissue anchor and configured to anchor to the ventricle wall.

In some instances, the first tissue anchor is configured to assume a compressed form within the lumen of the delivery shaft and assume an expanded form following removal from the lumen of the delivery shaft. The frame and membrane may have a diamond shape.

For purposes of summarizing the disclosure, certain aspects, advantages and novel features have been described. It is to be understood that not necessarily all such advantages may be achieved in accordance with any particular instance. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart having a leaflet anchor deployed therein in accordance with one or more examples.

FIG. 2 is a perspective view of a tissue anchor delivery device in accordance with one or more examples.

FIG. 3 illustrates an example tissue anchor configured to anchor one or more sutures and/or artificial chordae at one or more valve leaflets of a heart in accordance with one or more examples.

FIG. 4 illustrates another example tissue anchor comprising a pointed tip which may be configured to facilitate delivery of the anchor through one or more valve leaflets in accordance with one or more examples.

FIG. 5 illustrates a generally oval-shaped tissue anchor having a pointed tip to facilitate delivery of the anchor at one or more valve leaflets, in accordance with one or more instances.

FIG. 6 illustrates a droplet-shaped anchor configured for delivery and/or anchoring at one or more valve leaflets, in accordance with one or more instances.

FIG. 7 illustrates an example anchor following delivery at one or more valve leaflets, in accordance with one or more instances.

FIG. 8 illustrates an example anchor following delivery at one or more valve leaflets, in accordance with one or more instances.

FIGS. 9 (9-1, 9-2, and 9-3) provides a flow diagram illustrating a process for implanting a leaflet anchor in accordance with one or more examples.

FIGS. 10 (10-1, 10-2, 10-3, 10-4, and 10-5) provide images of cardiac anatomy and certain devices/systems corresponding to operations of the process of FIG. 9 in accordance with one or more examples.

FIG. 11 illustrates an example delivery system for delivering the one or more anchors described herein, in accordance with one or more instances.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular instance. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to certain device components/features and to the anatomy of animals, and namely humans, with respect to the preferred examples. Although certain spatially relative terms, such as “proximal,” “distal,” “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for deploying one or more anchors and/or attached neo chordae for chordal repair of various cardiac valves (e.g., the mitral and/or tri-cuspid valves) which may have experienced degenerative valve disease. One or more anchors described herein may comprise a stent-like structure configured to be compressed within a deployment device (e.g., a catheter). In some instances, an example anchor can comprise a membrane and/or weave portion configured to promote cellular overgrowth. One or more suture lengths may be configured to attach to an example anchor to allow for adjustment of a repaired valve leaflet. For example, the valve leaflet may be adjusted to reestablish leaflet coaptation and/or to minimize valve regurgitation.

The systems, devices, and methods described herein can allow for placement of multiple anchors and/or chordae utilizing a single delivery device and/or system and/or may eliminate the need to remove and/or reinsert a delivery device for subsequent deployments. As a result, a propensity for tangling of neo chordae may be minimized and/or a risk of damage to the neo chordae and/or native tissue due to chordae tangling may be minimized.

Delivery systems involving single-suture deployment devices can require an average of four to seven chordal replacements to be deployed per patient. This can require that multiple individual deployment devices be inserted into the beating heart, increasing the potential damage to the heart muscle, possible tangling of previously deployed chords with the subsequent chords, and/or a possibility of uneven loading and/or damaging of the neo chordae as a result of tangling and/or interference. The instances described herein can advantageously minimize these risks by allowing for simultaneous and/or single-procedure delivery of multiple tissue anchors and/or neo chordae. For example, example tissue anchors may be configured to be stacked within a single deployment device (e.g., a catheter). The tissue anchors may be advanced using a “ratchet” and/or similar mechanism to advance each anchor and/or suture.

Some tissue anchors described herein can comprise a pointed tip to allow the tissue anchors to independently penetrate leaflet tissue prior to deployment of the tissue anchors at one or more leaflets. The tissue anchors may be attached to sutures configured to allow for adjustment of a valve leaflet. In some instances, the deployment device can incorporate a suture management system that allows for free movement of each suture once deployed. Following deployment of one or more tissue anchors, the deployment device(s) may be removed from the patient's heart. Sutures attached to the anchors can be cinched and/or tensioned as a group and/or individually to achieve a desired coaptation of the valve leaflets and/or to minimize and/or eliminate valve regurgitation. Once properly adjusted, the suture ends may be anchored to the exterior wall of the heart and/or trimmed in order to complete valve repair.

Certain examples are disclosed herein in the context of cardiac implants and procedures. However, although certain principles disclosed herein are particularly applicable to the anatomy of the heart, it should be understood that puncture locator devices and puncture/access procedures in accordance with the present disclosure may be implanted in, or configured for implantation in, any suitable or desirable anatomy.

### Cardiac Physiology

The following includes a general description of human cardiac anatomy that is relevant to certain inventive features and examples disclosed herein and is included to provide context for certain aspects of the present disclosure. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

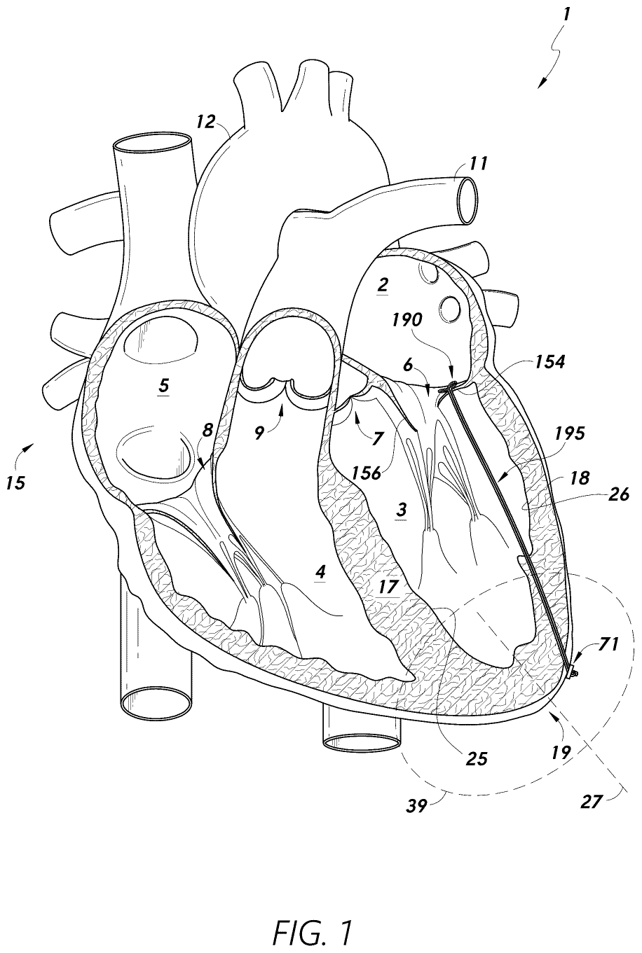

FIG. 1 illustrates an example representation of a heart 1 having various features relevant to certain aspects of the present inventive disclosure. The heart 1 includes four chambers, namely the left ventricle 3, the left atrium 2, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex and is generally located on the midclavicular line, in the fifth intercostal space. The apex 19 can be considered part of the greater apical region 39.

The left ventricle 3 is the primary pumping chamber of the heart 1. A healthy left ventricle is generally conical or apical in shape in that it is longer (along a longitudinal axis extending in a direction from the aortic valve 7 to the apex 19) than it is wide (along a transverse axis extending between opposing walls 25, 26 at the widest point of the left ventricle) and descends from a base 15 with a decreasing cross-sectional circumference to the point or apex 19. Generally, the apical region 39 of the heart is a bottom region of the heart that is within the left or right ventricular region but is distal to the mitral 6 and tricuspid 8 valves and toward the tip of the heart. More specifically, the apical region 39 may be considered to be within about 20 cm to the right or to the left of the median axis 27 of the heart 1.

The pumping of blood from the left ventricle is accomplished by a squeezing motion and a twisting or torsional motion. The squeezing motion occurs between the lateral wall 18 of the left ventricle and the septum 17. The twisting motion is a result of heart muscle fibers that extend in a circular or spiral direction around the heart. When these fibers contract, they produce a gradient of angular displacements of the myocardium from the apex 19 to the base 15 about the longitudinal axis of the heart. The resultant force vectors extend at angles from about 30-60 degrees to the flow of blood through the aortic valve 7. The contraction of the heart is manifested as a counterclockwise rotation of the apex 19 relative to the base 15, when viewed from the apex 19. A healthy heart can pump blood from the left ventricle in a very efficient manner due to the spiral contractility of the heart.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (e.g., mitral and tricuspid) heart valves may comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles (two shown in FIG. 1). The leaflets of the tricuspid valve may be referred to as the anterior, posterior and septal leaflets, respectively. The valve leaflets are connected to the papillary muscles by the chordae tendineae, which are disposed in the right ventricle 4 along with the papillary muscles.

Surrounding the ventricles (3, 4) are a number of arteries (not shown) that supply oxygenated blood to the heart muscle and a number of veins that return the blood from the heart muscle. The coronary sinus (not shown) is a relatively large vein that extends generally around the upper portion of the left ventricle 3 and provides a return conduit for blood returning to the right atrium 5. The coronary sinus terminates at the coronary ostium (not shown) through which the blood enters the right atrium.

With respect to the mitral valve 6, a normal mitral valve may comprise two leaflets (anterior and posterior) and two corresponding papillary muscles. The papillary muscles originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

Various disease processes can impair the proper functioning of one or more of the valves of the heart. These disease processes include degenerative processes (e.g., Barlow's disease, fibroelastic deficiency), inflammatory processes (e.g., rheumatic heart disease) and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in one or more leaflets of the valve which results in prolapse and regurgitation.

The mitral valve 6 and tricuspid valve 8 can be divided into three parts: an annulus, leaflets, and a sub-valvular apparatus. The sub-valvular apparatus can be considered to include the papillary muscles and the chordae tendineae, which can elongate and/or rupture. If a valve is functioning properly, when closed, the free margins or edges of the leaflets come together and form a tight junction, the arc of which, in the mitral valve, is known as the line, plane or area of coaptation. Normal mitral and tricuspid valves open when the ventricles relax allowing blood from the atrium to fill the decompressed ventricle. When the ventricle contracts, the chordae tendineae advantageously properly tether or position the valve leaflets such that the increase in pressure within the ventricle causes the valve to close, thereby preventing blood from leaking into the atrium and assuring that substantially all of the blood leaving the ventricle is ejected through the aortic valve 7 or pulmonic valve 9 and into the arteries of the body. Accordingly, proper function of the valves depends on a complex interplay between the annulus, leaflets, and sub-valvular apparatus. Lesions in any of these components can cause the valve to dysfunction and thereby lead to valve regurgitation.

Generally, there are three mechanisms by which a heart valve becomes regurgitant or incompetent; they include Carpentier's type I, type II and type III malfunctions. A Carpentier type I malfunction involves the dilation of the annulus such that normally functioning leaflets are distracted from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

One or more chambers in the heart 1 may be accessed in accordance with certain heart valve-repair procedures and/or other interventions. Access into a chamber in the heart may be made at any suitable site of entry. In some implementations, access is made to a chamber of the heart, such as a target ventricle (e.g., left ventricle) associated with a diseased heart valve, through the apical region 39. For example, access into the left ventricle 3 (e.g., to perform a mitral valve repair) may be gained by making a relatively small incision at the apical region 39, close to (or slightly skewed toward the left of) the median axis 27 of the heart. Access into the right ventricle 4 (e.g., to perform a tricuspid valve repair) may be gained by making a small incision into the apical region 39, close to or slightly skewed toward the right of the median axis 27 of the heart. Accordingly, the ventricle can be accessed directly via the apex, or via an off-apex location that is in the apical region 39 but slightly removed from the tip/apex, such as via lateral ventricular wall, a region between the apex and the base of a papillary muscle, or even directly at the base of a papillary muscle. In some implementations, the incision made to access the appropriate ventricle of the heart is no longer than about 1 mm to about 5 cm, from 2.5 mm to about 2.5 cm, or from about 5 mm to about 1 cm in length. When a percutaneous approach is sought, no incision into the apex region of the heart may be made, but rather access into the apical region 39 may be gained by direct needle puncture, for instance by an 18-gauge needle, through which an appropriate repair instrument can be advanced.

### Heart Valve Leaflet Tethering

Certain inventive features disclosed herein relate to certain heart valve repair systems and devices, and/or systems, process, and devices for repairing any other type of target organ tissue. In some implementations, a tissue anchor delivery device may be employed in repairing a mitral valve in patients suffering from degenerative mitral regurgitation or other condition. In some implementations, a transapical, off-pump repair procedure is implemented in which at least part (e.g., a shaft portion/assembly) of a valve repair system is inserted in the left ventricle and advanced to the surface of the diseased portion of a target mitral valve leaflet and used to deploy/implant a tissue anchor in the target leaflet. The tissue anchor may advantageously be integrated or coupled with one or more artificial/synthetic cords serving a function similar to that of chordae tendineae. Such artificial cord(s) may comprise suture(s) and/or suture tail portions associated with a knot-type tissue anchor and may comprise any suitable or desirable material, such as expanded polytetrafluoroethylene (ePTFE) or the like. The term “suture” is used herein according to its broad and ordinary meaning and may refer to any elongate cord, strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord,” “chord,” “chordae,” and “suture” may be used substantially interchangeably. In addition, use of the singular form of any of the suture-related terms listed above, including the terms “suture” and “cord,” may be used to refer to a single suture/cord, or to a portion thereof, or to a plurality of suture/cords, such as a pair of suture/cord tails emanating from a single anchor, knot, form, device, or other structure or assembly. Where a suture knot or anchor is deployed on a distal side of a tissue portion, and where two suture portions extend from the knot/anchor on a proximal side of the tissue, either or both of the suture portions may be referred to as a “suture” or a “cord,” regardless of whether both portions are part of a unitary suture or cord or are separate.

Processes for repairing a target organ tissue, such as repair of mitral valve leaflets to address mitral valve regurgitation, can include inserting a tissue anchor delivery device, such as a delivery device as described in PCT Application No. PCT/US2012/043761, (published as WO 2013/003228, and referred to herein as “the '761 PCT Application”) and/or in PCT Application No. PCT/US2016/055170 (published as WO 2017/059426 and referred to herein as “the '170 PCT Application”), the entire disclosures of which are incorporated herein by reference for all purposes, into a body and extending a distal end of the delivery device to a proximal side of the target tissue (e.g., leaflet).

The '761 PCT Application and the '170 PCT Application describe in detail methods and devices for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which may depend on the specific abnormality and the tissues involved.

With further reference to FIG. 1, FIG. 1 shows an example deployed leaflet/tissue anchor 190 deployed in a heart valve leaflet (e.g., a posterior leaflet 154 and/or anterior leaflet 156) and tethered to a heart/ventricle wall 18 via one or more sutures/suture tails 195 coupled to and/or associated with the anchor 190. The suture tails 195 coupled to the anchor 190 may be secured at the desired tension using a pledget 71 or other suture-fixing/locking device or mechanism on the outside of the heart wall 18 through which the suture tails 195 may run. A knot or other suture fixation mechanism or device may be implemented to hold the sutures at the desired tension and to the pledget 71. With the suture tail(s) 195 fixed to the ventricle wall 18, a portion of the suture tail(s) 195 disposed within the ventricle 3 may advantageously function as replacement leaflet cords (e.g., chordae tendineae) that are configured to tether the target leaflet 154 in a desired manner.

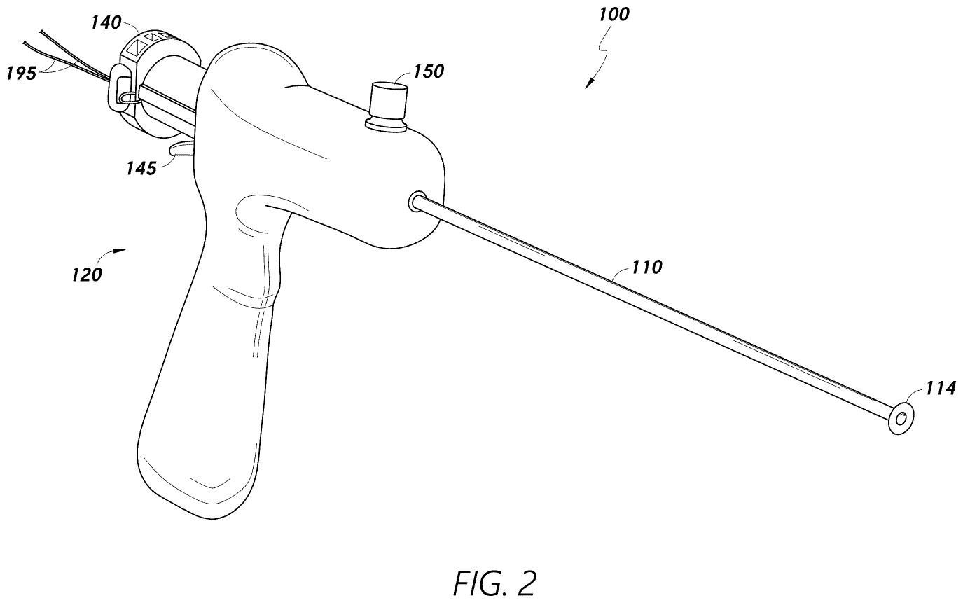

FIG. 2 is a perspective view of a tissue anchor delivery device in accordance with one or more examples. The tissue anchor delivery system 100 may be used to repair a heart valve, such as a mitral valve, and improve functionality thereof. For example, the tissue anchor delivery system 100 may be used to reduce the degree of mitral regurgitation in patients suffering from mitral regurgitation caused by, for example, midsegment prolapse of valve leaflets as a result of degenerative mitral valve disease. In order to repair such a valve, the tissue anchor delivery system 100 may be utilized to deliver and anchor tissue anchors, such as malleable tissue anchors, in a prolapsed valve leaflet. As described in detail below, such procedure may be implemented on a beating heart.

The delivery system 100 includes a rigid elongate tube 110 forming at least one internal working lumen. Although described in certain examples and/or contexts as comprising a rigid elongate tube, it should be understood that tubes, shafts, lumens, conduits, and the like disclosed herein may be either rigid, at least partially rigid, at least flexible, and/or at least partially flexible. Therefore, any such component described herein, whether or not referred to as rigid herein should be interpreted as possibly being at least partially flexible. In accordance with the present disclosure, the rigid elongate tube 110 may be referred to as a shaft for simplicity. Implementation of a valve-repair procedure utilizing the delivery system 100 can be performed in conjunction with certain imaging technology designed to provide visibility of the shaft 110 of the delivery system 100 according to a certain imaging modality, such as echo imaging. Generally, when performing a valve-repair procedure utilizing the tissue anchor delivery system 100, the operating physician may advantageously work in concert with an imaging technician, who may coordinate with the physician to facilitate successful execution of the valve-repair procedure.

In addition to the delivery shaft 110, the delivery system 100 may include a plunger feature 140. The tissue anchor delivery system 100 may further include a plunger lock mechanism 145, which may serve as a safety lock that locks the valve delivery system until ready for use or deployment of a leaflet anchor as described herein. The plunger 140 may have associated therewith a suture-release mechanism, which may be configured to lock in relative position a pair of suture tails 195 associated with a pre-formed knot anchor (not shown) to be deployed. For example, the suture portions 195 may be ePTFE sutures. The system 100 may further comprise a flush port 150, which may be used to de-air the lumen of the shaft 110. For example, heparinized saline flush, or the like, may be connected to the flush port 150 using a female Luer fitting to de-air the valve repair system 100. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure.

The lumen of the shaft 110 may house a needle (not shown) configured to deploy one or more anchors, as described in detail herein. In some examples, the shaft 110 presents a relatively low profile. For example, the shaft 110 may have a diameter of approximately 3 mm or less (e.g., about 9 Fr). The shaft 110 is associated with an atraumatic tip 114 feature. The atraumatic tip 114 can be an echogenic leaflet-positioner component, which may be used for deployment and/or positioning of the suture-type tissue anchor. The atraumatic tip 114, disposed at the distal end of the shaft 110, may be configured to have deployed therefrom one or more valve anchors, as described herein.

The atraumatic tip 114 may be referred to as an “end effector.” In addition to one or more valve anchors and associated needle, the shaft 110 may house an elongated knot pusher tube (not shown; also referred to herein as a “pusher”), which may be actuated using the plunger 140 in some examples. As described in further detail below, the tip 114 provides a surface against which the target valve leaflet may be held in connection with deployment of a leaflet anchor.

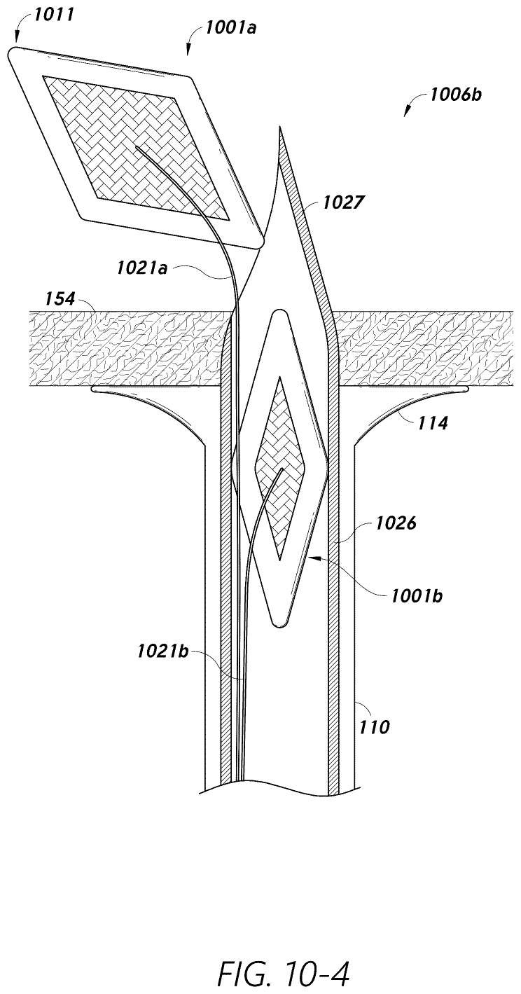

The delivery system 100 may be used to deliver any of the various tissue anchors described in greater detail below. For example, the delivery system 100 may be utilized to deliver a tissue anchor on a distal side of a mitral valve leaflet. The tip 114 (e.g., end effector), can be placed in contact with the ventricular side of a leaflet of a mitral valve. The tip 114 can be coupled to the distal end portion of the shaft 110, wherein the proximal end portion of the shaft 110 may be coupled to a handle portion 120 of the delivery system 100, as shown. Generally, the elongate pusher (not shown) may be movably disposed within a lumen of the shaft 110 and coupled to a pusher hub (not shown) that is movably disposed within the handle 120 and releasably coupled to the plunger 140. A needle and/or catheter carrying one or more tissue anchors can be movably disposed within a lumen of the pusher and coupled to a needle hub (not shown) that is also coupled to the plunger 140. In some instances, the needle and/or catheter may comprise a pointed tip to facilitate puncturing a valve leaflet. However, in some instances, one or more tissue anchors may have pointed tips and/or the needle and/or catheter may not comprise a pointed tip. The plunger 140 can be used to actuate or move the needle and/or the pusher during deployment of a distal anchor (see, e.g., FIG. 10) and is movably disposed at least partially within the handle 120. For example, the handle 120 may define a lumen in which the plunger 140 can be moved. During operation, the pusher may also move within the lumen of the handle 120. The plunger lock 145 can be used to prevent the plunger 140 from moving within the handle 120 during storage and prior to performing a procedure to deploy a tissue anchor.

The needle and/or catheter may have the one or more tissue anchors disposed at a distal portion thereof while maintained in the shaft 110. For example, one or more tissue anchors may be arranged generally longitudinally while within a lumen of the needle and/or catheter. In some instances, one or more suture tails may extend from each of the one or more tissue anchors. The suture tails 195 may extend through the lumen of the needle and/or through a passageway of the plunger 140 and may exit the plunger 140 at a proximal end portion thereof.

The delivery device/system 100 can further include a suture/tether catch mechanism (not shown) coupled to the plunger 140 at a proximal end of the delivery system 100, which may be configured to releasably hold or secure a suture 195 extending through the delivery system 100 during delivery of a tissue anchor as described herein. The suture catch can be used to hold the suture 195 with a friction fit or with a clamping force and can have a lock that can be released.

As described herein, the anchor delivery system 100 can be used in beating heart mitral valve repair procedures. In some instances, the shaft 110 of the delivery system 100 can be configured to extend and contract with the beating of the heart. During systolic contraction, the median axis of the heart generally shortens. For example, with reference to FIG. 1, the distance from the apex 19 of the heart to the valve leaflets 154, 154 can vary by about 1 cm to about 2 cm with each heartbeat in some patients. In some instances, the length of the shaft 110 that protrudes from the handle 120 can change with the length of the median axis of the heart. That is, distal end of the shaft 110 can be configured to be floating such that the shaft can extend and retract with the beat of the heart so as to maintain contact with the target mitral valve leaflet.

Advancement of the delivery system 100 may be performed in conjunction with echo imaging, direct visualization (e.g., direct transblood visualization), and/or any other suitable remote visualization technique/modality. With respect to cardiac procedures, for example, the delivery system 100 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). Typical procedures that can be implemented using echo guidance are set forth in Suematsu, Y., J. Thorac. Cardiovasc. Surg. 2005; 130:1348-56 (“Suematsu”), the entire disclosure of which is incorporated herein by reference.

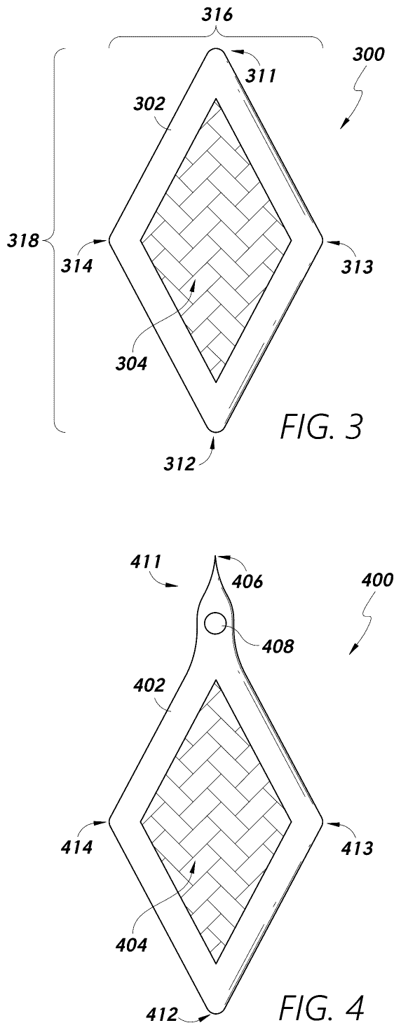

FIG. 3 illustrates an example tissue anchor 300 configured to anchor one or more sutures and/or artificial chordae at one or more valve leaflets of a heart. In some environments, the anchor 300 may have a diamond and/or rectangular shape and/or may comprise one or more corners and/or points, including a first point 311 across from a second point 312 and/or a third point 313 across from a fourth point 314. One or more of the points may be pointed ends and/or may represent a change in direction of an outer frame 302. The anchor 300 may comprise an outer frame 302 at least partially enclosing an at least partially flexible and/or porous membrane 304. For example, the outer frame 302 may at least partially surround a space having a shape similar to a shape of the outer frame 302. In the example shown in FIG. 3, the outer frame 302 may be diamond-shaped and/or may enclose a space that is similarly diamond-shaped. The membrane 304 may be configured to extend at least partially across an interior space and/or opening of the frame 302. In some instances, the frame 302 and/or membrane 304 may be at least partially composed of any of a variety of generally flexible materials. For example, the frame 302 may be at least partially composed of one or more shape memory alloys (e.g., nitinol) and/or the membrane may comprise a network of interwoven fibers and/or cords. In some instances, the frame 302 may be configured to bend such that at least the first point 311 extends at least partially over the membrane 304. The first point 311 and/or other points may have relatively sharp tips such that the first point 311 and/or other points may be configured to puncture a valve leaflet. However, the first point 311 and/or other points may additionally or alternatively have rounded and/or blunt tips and/or may not be configured to puncture a valve leaflet.

In some instances, the anchor 300 may have a stent-like structure and/or may be movable between a compressed and/or expanded form. For example, the anchor 300 may be configured to assume a compressed form while within a catheter and/or other delivery device. In the compressed form, the anchor 300 may assume an at least partially modified form from the default and/or expanded form shown in FIG. 3. For example, compression of the anchor may involve a decrease of a width 316 of the anchor 300 and/or an increase of a length 318 of the anchor 300 as the frame 302 bends in response to outside forces. Upon removal from the delivery device, the anchor 300 may be configured to naturally expand and/or to be expanded using wires and/or other manual means. Expansion of the anchor may involve an increase in the width 316 and/or a decrease in the length 318. The width 316 may represent a distance between the third point 313 and the fourth point 314. The length 318 may represent a distance between the first point 311 and the second point 312.

The membrane 304 may comprise a network of weaved and/or interwoven materials, which can include various cords and/or fibers. In some instances, the membrane 304 may be configured to promote cellular overgrowth following deployment at a valve leaflet and/or other target area. For example, the membrane 304 may have a generally porous structure and/or may be configured to allow tissue growth through openings and/or gaps of the membrane 304. The anchor 300 may be configured to lay generally flatly along a surface of a valve leaflet such that the membrane 304 may extend over a surface area of the valve leaflet.

In some instances, one or more sutures and/or similar devices may be configured to couple, attach, and/or extend from the anchor 300. For example, a suture may be configured to couple to the anchor 300 to allow for adjustment of a valve leaflet which the anchor 300 may be anchored to. Adjustment of the valve leaflet may be configured to cause and/or reestablish leaflet coaptation and/or to minimize valve regurgitation. The one or more sutures may be configured to anchor and/or attach to any portion of the anchor 300. For example, a suture may be configured to attach to a central portion of the membrane 304 of the anchor 300. In this way, the suture may be configured to apply force across a wide area of the membrane 304 and/or frame 302.

The membrane 304 may be composed of any of a variety of suitable materials. For example, the membrane 304 may be at least partially composed of electro spun fabric. The membrane 304 may be configured to lay flatly against a tissue surface and/or to at least partially cover a delivery puncture through the tissue. The membrane 304 may comprise a closed-cell network of materials. In some instances, the membrane 304 may be configured to allow tissue growth through the membrane 304. For example the membrane 304 may have a porous structure including gaps between fibers forming the membrane 304.

The frame 302 may have a generally rigid and/or flexible structure. In some instances, the frame 302 may be configured to bend during deployment and/or following deployment. Bending of the frame 302 may allow for adjustments to the shape of the anchor 300. While the anchor 300 (e.g., the frame 302 and/or membrane 304) is shown having a diamond shape in FIG. 3, the anchor 300 may have any suitable shape. The anchor 300 is shown in FIG. 3 from an overhead view. For example, the anchor 300 (e.g., the frame 302 and/or membrane 304) may be configured to lay flatly against a tissue valve leaflet with at least a portion of the frame 302 and/or membrane 304 contacting and/or covering the tissue. Following deployment at the valve leaflet, the anchor 300 may be configured to cover a diamond-shaped area of the valve leaflet. However, the anchor 300 may be adjusted to change a coverage area of the anchor 300. For example, the first point 311 may be redirected to extend at least partially over the membrane 304 and/or over a puncture opening through the valve leaflet. Following adjustment of the anchor 300, the anchor may be configured to have a heart shape and/or to cover a heart-shaped area of the valve leaflet.

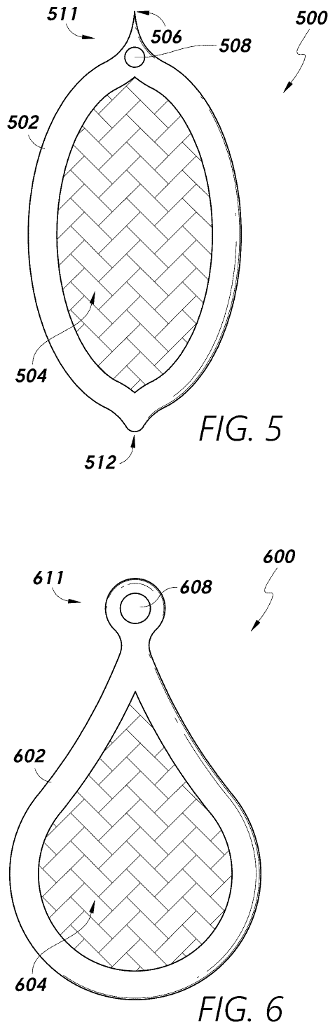

FIG. 4 illustrates another example tissue anchor 400 comprising a pointed tip 406 which may be configured to facilitate delivery of the anchor 400 through one or more valve leaflets. As shown in FIG. 4, one or more corners of the anchor 400 may be extended to form the pointed tip 406, which may be configured to penetrate a tissue wall and/or in valve leaflet. For example, the anchor 400 may comprise a frame 402 forming a first point 411 (e.g., corner) across from a second point 412 and/or a third point 413 across from a fourth point 414. The first point 411 may extend into the pointed tip 406 and/or may comprise an eyelet 408 (e.g., aperture) configured to receive one or more sutures and/or similar devices.

In some instances, the pointed tip 406 of the anchor 400 may be configured to facilitate delivery of the anchor 400. After the anchor 400 is extended through a valve leaflet, the anchor 400 may be deployed from a catheter and/or other delivery systems. In some instances, the anchor 400 may comprise one or more eyelets 408 configured to receive one or more sutures which may be tethered to the anchor 400. The eyelet 408 may comprise a generally circular opening in the first point 411 configured to allow one or more sutures to extend through the first point 411.

After the anchor 400 and/or the pointed tip 406 punctures through a valve leaflet, the anchor 400 may be configured to twist and/or adjust such that the pointed tip 406 extends along the valve leaflet such that the membrane 404 of the anchor lays flatly along the valve leaflet. For example, the anchor 400 may be configured to cover a diamond-shaped area of the valve leaflet. However, the anchor 400 may be adjusted to change a coverage area of the anchor 400. For example, the first point 411 may be redirected to extend at least the pointed tip 406 at least partially over the membrane 404 and/or over a puncture opening through the valve leaflet. Following adjustment of the anchor 400, the anchor may be configured to have a heart shape and/or to cover a heart-shaped area of the valve leaflet.

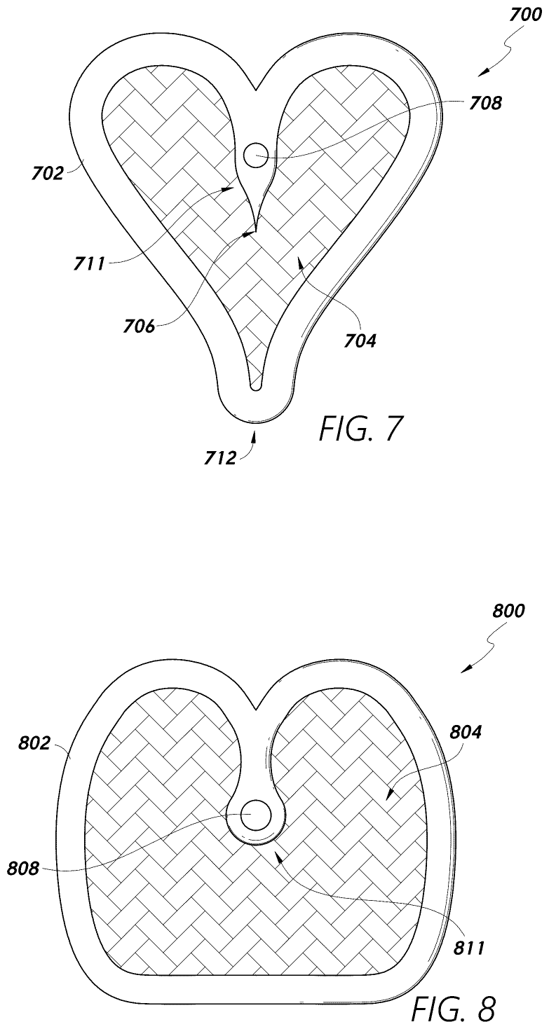

FIG. 5 illustrates a generally oval-shaped tissue anchor 500 having a pointed tip 506 to facilitate delivery of the anchor 500 at one or more valve leaflets, in accordance with one or more instances. The anchor 500 may comprise a generally oval-shaped frame 502 at least partially enclosing a generally oval-shaped membrane 504. The frame 502 may comprise one or more points and/or corners, which may include a first point 511 situated generally across from a second point 512. The first point 511 may extend into the pointed tip 506 and/or may comprise an eyelet 508 configured to receive one or more sutures and/or similar devices.

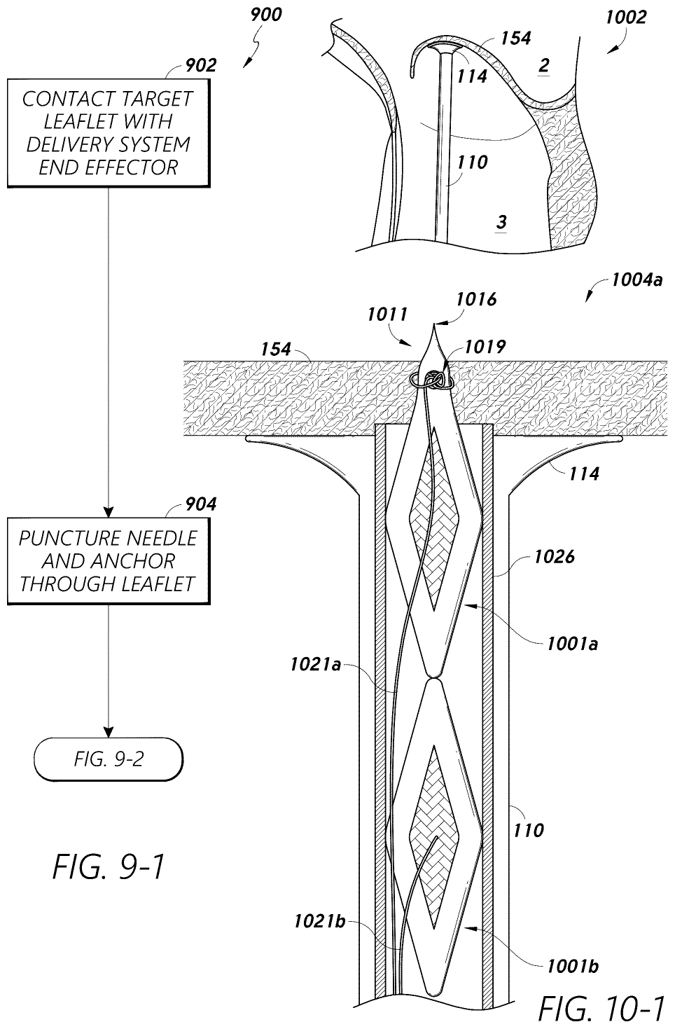

FIG. 6 illustrates a droplet-shaped anchor 600 configured for delivery and/or anchoring at one or more valve leaflets, in accordance with one or more instances. The anchor 600 may comprise a generally oval-shaped frame 602 at least partially enclosing a generally oval-shaped membrane 604. The frame 602 may comprise one or more points and/or corners, which may include a first point 611. The first point 611 may comprise an eyelet 608 configured to receive one or more sutures and/or similar devices.

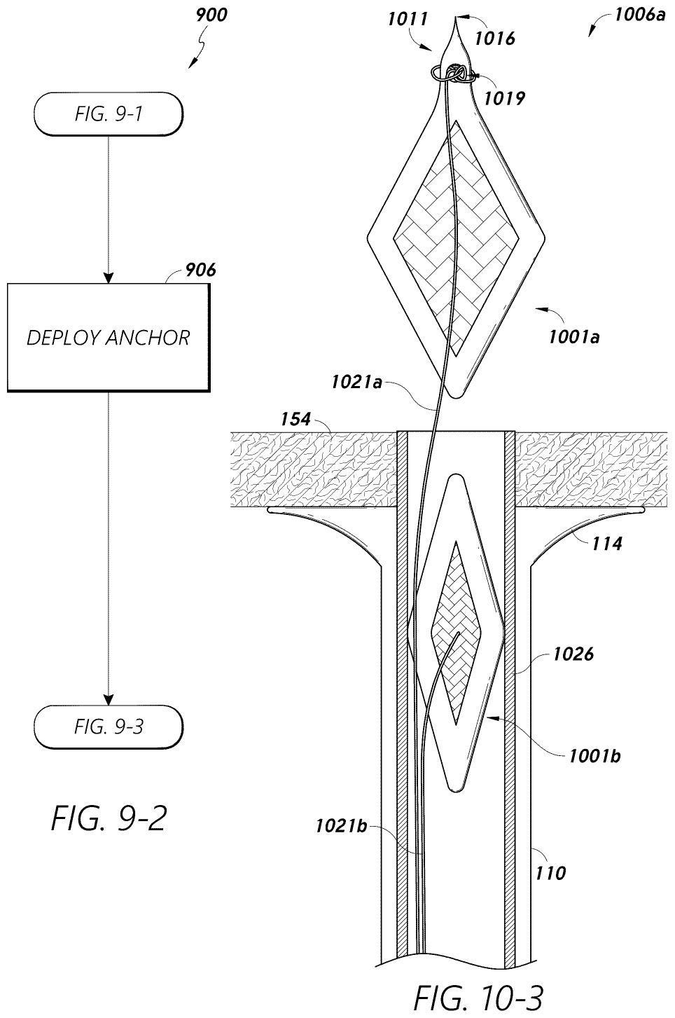

FIG. 7 illustrates an example anchor 700 following delivery at one or more valve leaflets, in accordance with one or more instances. The anchor 700 may comprise a frame 702 at least partially enclosing a membrane 704. In some instances, the frame 702 may comprise one or more points and/or anchors, which can include a first point 711 and/or a second point 712. The first point 711 may extend into a pointed tip 706 and/or may comprise an eyelet 708. The anchor 700 may be configured to assume the form shown in FIG. 7 following delivery at a valve leaflet. For example, the anchor 700 may comprise a diamond and/or oval form (see, e.g., FIGS. 4 and 5) during delivery of the anchor 700 through and/or against the valve leaflet. Following delivery of the anchor 700, the frame 702 of the anchor 700 may be configured to bend such that a pointed tip 706 of the frame 702 is redirected and/or extends towards the second point 712 and/or at least partially over the membrane 704. In some instances, the frame 702 may be configured to bend such that an eyelet 708 at the first point 711 may be situated approximately over a central portion of the membrane 704.

Redirecting the pointed tip 706 may cause a change of shape and/or form of the anchor 700. For example, a diamond and/or oval anchor 700 may assume a heart-shaped and/or similar form, as shown in FIG. 7. By extending the pointed tip 706 at least partially over the membrane 704, risk of damage from the pointed tip 706 to the surrounding tissue may be reduced.

As the frame 702 bends to allow redirection of the first point 711, the membrane 704 may be configured to bend, collapse, and/or fold in response to the movement of the frame 702. At least some portions of the membrane 704 may be configured to overlap with each other and/or a coverage area of the membrane 704 may be decrease as a result of bending of the frame 702.

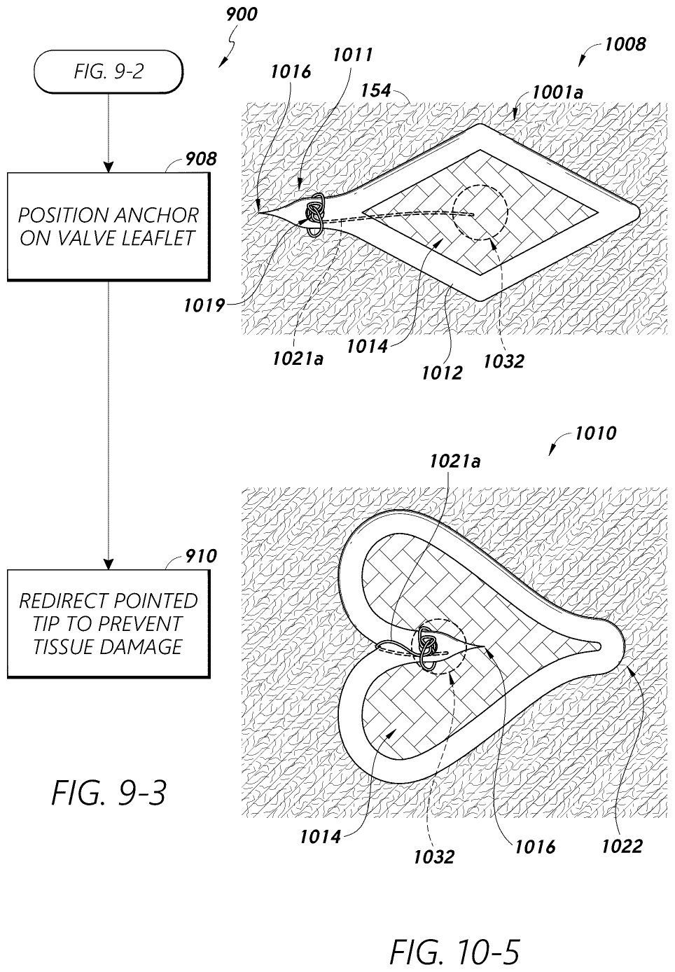

FIG. 8 illustrates an example anchor 800 following delivery at one or more valve leaflets, in accordance with one or more instances. The anchor 800 may comprise a frame 802 at least partially enclosing a membrane 804. In some instances, the frame 802 may comprise one or more points and/or anchors, which can include a first point 811. The first point 811 may comprise an eyelet 808. The eyelet 808 may be configured to receive one or more sutures configured to tether the anchor 800 to a second anchoring point within the point (e.g., a ventricle wall). For example, a suture may be configured to be knotted through the eyelet 808 to establish a secure attachment between the suture and the anchor 800.

The anchor 800 may be configured to assume the form shown in FIG. 8 following delivery at a valve leaflet. For example, the anchor 800 may comprise a droplet form (see, e.g., FIG. 6) during delivery of the anchor 800 through and/or against the valve leaflet. Following delivery of the anchor 800, the frame 802 of the anchor 800 may be configured to bend such that a first point 811 of the frame 802 is redirected and/or extends at least partially over the membrane 804. In some instances, the frame 802 may be configured to bend such that an eyelet 808 at the first point 811 may be situated approximately over a central portion of the membrane 804.

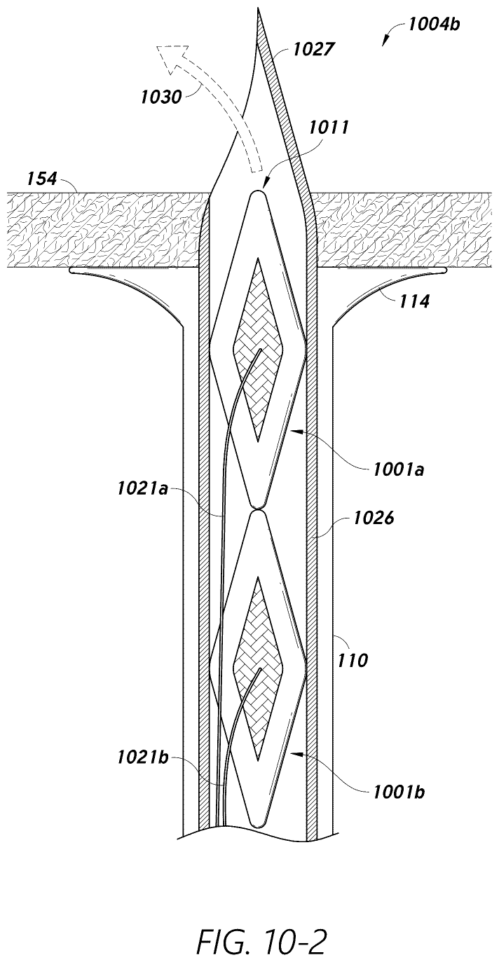

FIGS. 9 (9-1, 9-2, and 9-3) provides a flow diagram illustrating a process 900 for implanting a leaflet anchor in accordance with one or more examples. FIGS. 10 (10-1, 10-2, 10-3, 10-4, and 10-5) provide images of cardiac anatomy and certain devices/systems corresponding to operations of the process of FIG. 9 in accordance with one or more examples.

The process 900 may be implemented when a minimally invasive approach is determined to be advisable. Although not shown specifically in the flow diagram of FIG. 9, the process 900 may initially involve making one or more incisions proximate to the thoracic cavity to provide a surgical field of access. The total number and length of the incisions to be made depend on the number and types of the instruments to be used as well as the procedure(s) to be performed. The incision(s) may advantageously be made in such a manner as to be minimally invasive. As referred to herein, the term “minimally invasive” means in a manner by which an interior organ or tissue may be accessed with relatively little damage being done to the anatomical structure through which entry is sought. For example, a minimally invasive procedure may involve accessing a body cavity by a small incision of, for example, approximately 5 cm or less made in the skin of the body. The incision may be vertical, horizontal, or slightly curved. If the incision is located along one or more ribs, it may advantageously follow the outline of the rib. The opening may advantageously extend deep enough to allow access to the thoracic cavity between the ribs or under the sternum and is preferably set close to the rib cage and/or diaphragm, dependent on the entry point chosen.

In one example method, the heart may be accessed through one or more openings made by one or more small incision in a portion of the body proximal to the thoracic cavity, such as between one or more of the ribs of the rib cage of a patient, proximate to the xyphoid appendage, or via the abdomen and diaphragm. Access to the thoracic cavity may be sought to allow the insertion and use of one or more thorascopic instruments, while access to the abdomen may be sought to allow the insertion and use of one or more laparoscopic instruments. Insertion of one or more visualizing instruments may then be followed by transdiaphragmatic access to the heart. Additionally, access to the heart may be gained by direct puncture (e.g., via an appropriately sized needle, for instance an 18-gauge needle) of the heart from the xyphoid region. Accordingly, the one or more incisions should be made in such a manner as to provide an appropriate surgical field and access site to the heart in the least invasive manner possible. Access may also be achieved using percutaneous methods, further reducing the invasiveness of the procedure. See, e.g., “Full-Spectrum Cardiac Surgery Through a Minimal Incision Mini-Sternotomy (Lower Half) Technique,” Doty et al., Annals of Thoracic Surgery 1998; 65(2): 573-7 and “Transxiphoid Approach Without Median Sternotomy for the Repair of Atrial Septal Defects,” Barbero-Marcial et al., Annals of Thoracic Surgery 1998; 65 (3): 771-4, the entire disclosures of each of which are incorporated herein by reference.

At block 902, the process 900 involves contacting a target leaflet 154 with an end effector 114 of a delivery system, as shown in image 1002 of FIG. 10. Image 1002 shows the shaft 110 of the tissue anchor delivery system 100 positioned on the target valve leaflet 154 (e.g., mitral valve leaflet). For example, the target site of the valve leaflet 154 may be slowly approached from the ventricle side thereof by advancing the distal end of the shaft 110 along or near to the posterior wall of the ventricle 3 (e.g., left ventricle), without contacting the ventricle wall.

Once the tip 114 is positioned in the desired position, the distal end of the shaft 110 and the tip 114 may be used to drape, or “tent,” the leaflet 154 to better secure the tip 114 in the desired position, as shown in image 1002. Draping/tenting may advantageously facilitate contact of the tip 114 with the leaflet 154 throughout one or more cardiac cycles, to thereby provide more secure or proper deployment of leaflet anchor(s). The target location may advantageously be located relatively close to the free edge of the target leaflet 154 to minimize the likelihood of undesirable intra-atrial wall deployment of the anchor. Navigation of the tip 114 to the desired location on the underside of the target valve leaflet 154 may be assisted using echo imaging, as described in detail herein. Echo imaging may be relied upon to confirm correct positioning of the tip 114 prior to anchor/knot deployment.

At block 904, the process 900 involves puncturing the valve leaflet 154 using one or more anchors 1001 and/or catheters 1026 (including needles and/or other tipped delivery systems). For example, as shown in image 1004a, a first anchor 1001a may comprise a pointed tip 1016 at a first point 1011 of the first anchor 1001a. The pointed tip 1016 may be configured to extend at least partially beyond the needle 1026 to contact and/or puncture a proximal surface of the valve leaflet 154. The pointed tip 1016 of the first anchor 1001a may advantageously allow for delivery of one or more anchors 1001 without requiring delivery systems comprising a needle tip. While only the first anchor 1001a is shown comprising a pointed tip 1016, a second anchor 1001b and/or any additional anchors 1001 may also comprise pointed tips 1016 for puncturing other areas of the valve leaflet 154.

As shown in images 1004a and 1004b, the needle 1026 may be configured to carry multiple anchors 1001 in a stacked configuration to allow for deployment of multiple anchors 1001 via the needle 1026. While the anchors 1001 are shown situated in an end-to-end manner, the anchors 1001 may be situated in any suitable manner within the needle 1026. In some instances, one or more pushers and/or ratchet mechanisms may be used to deploy the anchors 1001 from the needle 1026 one at a time.

In some instances, the needle 1026 may comprise a needle tip 1027, as shown in image 1004b. The needle tip 1027 may be radiused and/or may extend over a midpoint and/or lumen of the needle 1026 such that the needle tip 1027 may be configured to puncture a portion of the valve leaflet 154 that is situated over a midpoint and/or lumen of the needle 1026. Edges of the needle 1026 along the beveled and/or pointed end of the needle 1026 may be rounded and/or may be configured to cause dilation of the valve leaflet 154 rather than cutting of the valve leaflet 154. For example, a width of the needle tip 1027 may increase from the pointed end to the length of the shaft 110. Thus, as the needle 1026 is extended further into and/or through the leaflet 154, the needle tip 1027 may gradually increase a width and/or size of a puncture opening through the leaflet 154. In some instances, the beveled needle tip 1027 of the needle 1026 may be configured to deploy the one or more anchors 1001 at an angle 1030 (e.g., at an approximately 45-degree angle) from the inner lumen of the needle 1026. By deploying the one or more anchors 1001 at an angle 1030, each anchor 1001 may advantageously be deployed out of the way of any subsequent anchors 1001. For example, the needle 1026 may be twisted to adjust a deployment position of the anchors. Where the needle 1026 comprises a needle tip 1027, the one or more anchors 1001 may not comprise pointed tips 1016.

Each of the one or more anchors 1001 may have one or more sutures 1021 attached to and/or extending from the anchor 1001. In some instances, one or more anchors 1001 (e.g., the first anchor 1001a) may comprise an eyelet configured to receive one or more sutures 1021. For example, a first suture 1021a may be configured to extend through an eyelet of the first anchor 1001a and/or to form a knot 1019 at or near the eyelet to secure the first suture 1021a to the first anchor 1001a. For anchors 1001 that do not comprise an eyelet, one or more sutures 1021 may be configured to attach to a membrane of the anchors (see, e.g., the second suture 1021b attaching to the second anchor 1001b). Each of the anchors 1001 may be coupled and/or tethered to a different suture 1021. The sutures 1021 may each extend from the anchors 1001 through the lumen of the shaft and/or may be configured to be anchored to a pledget and/or otherwise at a tissue wall to provide tension to the anchors 1001.

With the shaft 110 positioned against the target leaflet 154, a plunger of the tissue anchor delivery device can be actuated to move the needle 1026 and a pusher disposed within the shaft 110. As the plunger 140 is actuated, a distal piercing portion of the anchor 1001 and/or needle 1026 punctures the leaflet 154 and forms an opening in the leaflet. In some instances, the anchor 1001 and/or needle 1026 is projected a distance of between about 0.2-0.3 inches (e.g., between about 5-8 mm), or less, distally beyond the distal end of the shaft 110 (e.g., beyond the tip 114). In some instances, the anchor 1001 and/or needle 1026 is projected a distance of between about 0.15-0.4 inches (e.g., between about 3-10 mm). In some instances, the anchor 1001 and/or needle 1026 is projected a distance of about 1 inch (e.g., about 2.5 cm), or greater. In some instances, the needle 1026 extends until the anchor 1001 and/or needle 1026 extend through the leaflet 154. While the anchor 1001 and/or needle 1026 are projected into the atrial side of the leaflet 154, the shaft 110 and tip 114 advantageously remain entirely on the ventricular side of the leaflet 154.

As the pusher (not shown) within the tissue anchor delivery device shaft 110 is moved distally, a distal end of the pusher advantageously moves or pushes the first anchor 1001a over the distal end of the needle 1026 and further within the atrium of the heart on a distal side of the leaflet 154, such that the first anchor 1001a extends distally beyond a distal end of the needle 1026.

At block 906, the process 900 involves deploying at least a first anchor 1001a beyond a distal end of the needle 1026, through the puncture opening of the valve leaflet 154, and/or beyond a distal surface of the valve leaflet 154, as shown in images 1006a and 1006b of FIG. 10. In some instances, the anchors 1001 may be deployed one at a time and/or a single anchor 1001 may be deployed at each puncture site. Following deployment of the first anchor 1001a beyond the distal surface of the valve leaflet 154, the needle 1026 may be moved to allow for deployment of the second anchor 1001b at a different portion of the valve leaflet 154.

In some instances, the first anchor 1001a and/or second anchor 1001b may be configured to assume a compressed form while within the lumen of the needle 1026 and/or shaft 110. For example, the walls of the needle 1026 may press against the sides of the first anchor 1001a and/or second anchor 1001b. The first anchor 1001a and/or second anchor 1001b may be configured to compress laterally and/or extend longitudinally (e.g., along the needle 1026) in response to pressure from the walls of the needle 1026. Following removal from the lumen of the needle 1026, the first anchor 1001a and/or second anchor 1001b may be configured to assume an expanded in form, in which the first anchor 1001a and/or second anchor 1001b may expand laterally and/or compress longitudinally. The first anchor 1001a and the second anchor 1001b may be configured to be situated end-to-end longitudinally within the needle 1026.

At block 908, the process 900 involves positioning the first anchor 1001a on the valve leaflet 154, as shown in image 1008 of FIG. 10. Following removal of the first anchor 1001a from the needle 1026, the first anchor 1001a may be configured to twist and/or to be twisted such the first anchor 1001a extend generally perpendicularly from the delivery needle 1026 and/or along a surface of the valve leaflet 154. For example, the first anchor 1001a may be twisted such that the membrane 1014 of the first anchor 1001a extends along and/or contacts the distal surface of the valve leaflet 154. The first anchor 1001a may be configured to cover a generally diamond-shaped and/or other shaped portion of the valve leaflet 154. The membrane 1014 may be configured to at least partially cover the puncture 1032 and/or puncture opening through the valve leaflet 154. The first suture 1021a may extend through the puncture 1032 while the first anchor 1001a remains situated at the distal surface of the valve leaflet 154.

At block 910, the process 900 involves redirecting a first point 1011 and/or pointed tip 1016 of the first anchor 1001a to prevent damage to the valve leaflet 154, as shown in image 1010 of FIG. 10. The first point 1011 and/or pointed tip 1016 may be redirected toward a second point 1022 of the frame 1012 and/or at least partially along the membrane 1014. Following redirection of the first point 1011 and/or pointed tip 1016, the pointed tip 1016 and/or eyelet may be situated at least partially over the puncture 1032 and/or membrane 1014.

Steps of the process 900 may be repeated for other anchors 1001 delivered via the shaft 110. For example, as the first anchor 1001a is deployed beyond the distal end of the shaft 110, the second anchor 1001b may take the place of the first anchor 1001a and/or may be pushed toward the distal end of the shaft 110. Following deployment of the first anchor 1001a, the shaft 110 and/or needle 1026 may be repositioned and/or placed below a different portion of the leaflet 154. The needle 1026 and/or second anchor 1001b may then be used to create a second puncture opening in the leaflet 154. The second anchor 1001b may be extended beyond the distal end of the needle 1026 and/or may otherwise be deployed at a distal side of the leaflet 154. The second anchor 1001b may be adjusted such that a membrane of the second anchor 1001b at least partially covers the second puncture opening. In some instances, the second anchor 1001b may be at least partially flexible to allow adjusting the second anchor 1001b such that at least a first point of the second anchor 1001b extends at least partially along the membrane of the second anchor 1001b and/or at least partially over the second puncture opening.

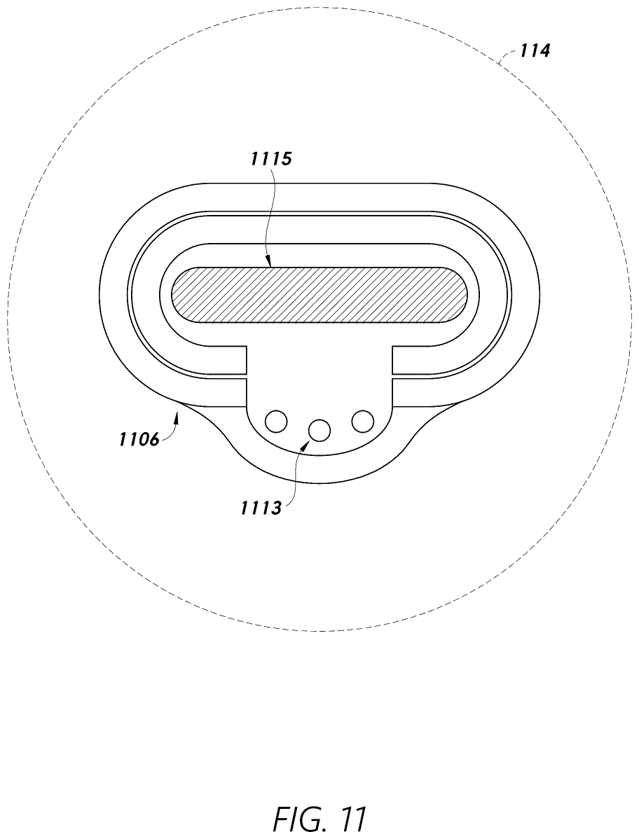

FIG. 11 illustrates an example delivery system for delivering the one or more anchors described herein, in accordance with one or more instances. In some instances, a shaft 1106 and/or catheter 1115 may comprise one or more receptors 1113 and/or suture channels configured to receive one or more sutures extending from one or more anchors. For example, one or more anchors may be configured to be carried and/or deployed via the shaft 1106 and/or catheter 1115. Sutures attached to the anchors may be configured to each extend into one of multiple receptors 1113 of the delivery system to prevent tangling of the sutures. While three receptors 1113 are shown in FIG. 11, the delivery systems may comprise any number of receptors 1113. The delivery systems may also comprise an atraumatic tip for facilitating delivery of the one or more anchors and/or sutures.

In some implementations, echo imaging, such as involving TEE (two-dimensional (2D) and/or three-dimensional (3D)), transthoracic echocardiography (TTE), ICE, and/or cardio-optic direct visualization (e.g., via infrared vision from the tip of a 7.5 F catheter), or other imaging modality, may be performed to assess the heart, heart valves, and/or tissue anchor delivery device component(s) in connection with any of the steps of the processes described herein. For example, echo imaging can be used to guide positioning of tissue anchor(s) onto a target valve leaflet. Although the procedures described herein are with reference to repairing a cardiac mitral valve or tricuspid valve by the implantation of one or more leaflet anchors and associated cord(s), the methods presented are readily adaptable for various types of tissue, leaflet, and annular repair procedures. The methods described herein, for example, can be performed to selectively approximate two or more portions of tissue to limit a gap between the portions. That is, in general, the methods herein are described with reference to a mitral valve but should not be understood to be limited to procedures involving the mitral valve.

### ADDITIONAL EXAMPLES

Depending on the instance, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular instance. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single instance, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular instance herein can be applied to or used with any other instance(s). Further, no component, feature, step, or group of components, features, or steps are necessary or indispensable for each instance. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A tissue anchor comprising:
a frame comprising at least a first pointed end; and
a membrane extending across an opening of the frame;
wherein the frame is configured to bend such that the first pointed end extends at least partially over the membrane.

2. The tissue anchor of claim 1, wherein the frame and membrane are diamond shaped.

3. The tissue anchor of claim 1, wherein the frame and membrane are oval-shaped.

4. The tissue anchor of claim 1, wherein the frame and membrane are droplet shaped.

5. The tissue anchor of claim 1, wherein the first pointed end is configured to puncture a valve leaflet of a heart.

6. The tissue anchor of claim 1, wherein the first pointed end comprises an eyelet configured to receive a tethering suture.

7. The tissue anchor of claim 1, wherein the membrane is configured to attach to a tethering suture.

8. The tissue anchor of claim 1, wherein the frame is configured to bend such that the frame assumes a heart-shaped form.

9. A method comprising:
delivering a needle carrying two or more tissue anchors situated longitudinally within a lumen of the needle, each of the two or more tissue anchors comprising a frame having a pointed end and a membrane extending across an opening of the frame, and each of the two or more tissue anchors tethered to a different suture of two or more sutures;
deploying a first tissue anchor of the two or more tissue anchors through a puncture opening in a valve leaflet of a heart and beyond a distal end of the needle;
positioning the first tissue anchor such that a first membrane of the first tissue anchor covers the puncture opening; and
redirecting a first pointed end of the frame of the first tissue anchor such that the pointed end extends at least partially over the first membrane.

10. The method of claim 9, further comprising puncturing the valve leaflet using a pointed tip of the needle.

11. The method of claim 10, wherein the pointed tip extends at least partially over the lumen of the needle to cause the first tissue anchor to exit the lumen at an angle with respect to the needle.

12. The method of claim 10, wherein the pointed tip has rounded edges to cause dilation of the puncture opening.

13. The method of claim 9, further comprising deploying a second tissue anchor of the two or more tissue anchors through a second puncture opening in the valve leaflet of the heart and beyond the distal end of the needle.

14. The method of claim 9, wherein redirecting the first pointed end of the frame causes the first pointed end to extend at least partially over the puncture opening.

15. A tissue anchoring system comprising:
a first tissue anchor configured for delivery via a lumen of a delivery shaft to a valve leaflet of a heart, the first tissue anchor comprising:
a frame comprising at least a first pointed end;
a membrane extending across an opening of the frame, wherein the frame is configured to bend such that the first pointed end extends at least partially over the membrane; and
a first suture tethered to the first tissue anchor and configured to anchor to a ventricle wall.

16. The tissue anchoring system of claim 15, wherein the frame comprises an eyelet, and wherein the first suture is configured to form a knot through the eyelet.

17. The tissue anchoring system of claim 15, wherein the first suture is configured to attach to the membrane.

18. The tissue anchoring system of claim 15, further comprising:
a second tissue anchor configured for delivery via the lumen of the delivery shaft to the valve leaflet of the heart; and
a second suture tethered to the second tissue anchor and configured to anchor to the ventricle wall.

19. The tissue anchoring system of claim 15, wherein the first tissue anchor is configured to assume a compressed form within the lumen of the delivery shaft and assume an expanded form following removal from the lumen of the delivery shaft.

20. The tissue anchoring system of claim 15, wherein the frame and membrane have a diamond shape.
